(19) United States
(12) Patent Application Publication
(10) Pub. No.: US 2011/0093039 A1
(43) Pub. Date: Apr. 21, 2011

(54) SCHEDULING INFORMATION DELIVERY TO A RECIPIENT IN A HEARING PROSTHESIS

(76) Inventor: Koen Van Den Heuvel, Hove (BE)

(21) Appl. No.: 12/988,512

(22) PCT Filed: Apr. 17, 2009
(86) PCT No.: PCT/AU09/00483

§ 371 Date: Jan. 5, 2011

(30) Foreign Application Priority Data
Apr. 17, 2008 (AU) 2008902011

(51) Int. Cl. A61N 1/05

(52) U.S. Cl. 607/57

(57) ABSTRACT

A hearing prosthesis for use by a recipient. The hearing prosthesis includes a receiver configured to receive sounds external to the recipient, a stimulator configured to stimulate tissue of the recipient to enhance recipient hearing, a sound analyzer configured to analyze the sounds received by the receiver, the sound analyzer further being configured to output a sound analysis signal indicative of the analyzed sounds, an information signal generator configured to output an information signal upon which an inputted indication that is provided to the recipient via the stimulator may be based, and an information scheduler configured to control the time at which the information signal is delivered to the stimulator based on the sound analysis signal.

### CROSS-REFERENCE TO RELATED APPLICATIONS

This present application is a National Stage Application of International Application No. PCT/AU2009/000483, entitled “Sound Processor for a Medical Implant”, filed on Apr. 17, 2009 which claims priority from Australian Patent Application No. 2008902011 entitled “Sound Processor for a Medical Implant”, filed on Apr. 17, 2008, both of which are hereby incorporated by reference herein.

### BACKGROUND

1. Field of the Invention

The present invention generally relates to hearing prostheses for recipients and, more specifically, to scheduling information delivery to a recipient using a hearing prosthesis.

2. Related Art

A variety of modern hearing prostheses are available for a recipient. Such hearing prostheses include, but are not limited to, cochlear implants, acoustical hearing aids, bone conduction devices, middle-ear implants, scala timpani stimulators, auditory brain stimulators, etc. Cochlear implants may be used to enhance hearing in a recipient who is completely deaf, while acoustical hearing aids may be used to enhance hearing in a recipient who has some residual hearing, but has difficulty hearing. So-called hybrid devices include a combination of, for example, a cochlear implant and an acoustical hearing aid. Often, the cochlear implant is used to enhance hearing at the higher frequencies while an acoustical hearing aid is used to enhance hearing at the lower frequencies (often, a human looses the ability to hear higher frequencies while still retaining the ability to hear lower frequencies).

Modern hearing prostheses may provide the ability to not only enhance hearing of sounds external to the recipient (e.g., speech, noise, music, etc.), but also the ability to permit a recipient to perceive sounds that are not external to the recipient (e.g., music resulting from signals from an MP3 directly transmitted to the hearing prosthesis, synthesized noises, etc.)

### SUMMARY

According to a first aspect of the present invention, there is a hearing prosthesis for use by a recipient to enhance hearing, the hearing prosthesis comprises a receiver configured to receive sounds external to the recipient, a stimulator configured to stimulate tissue of the recipient to enhance recipient hearing, a sound analyzer configured to analyze the sounds received by the receiver, the sound analyzer further being configured to output a sound analysis signal indicative of the analyzed sounds, an information signal generator configured to output an information signal upon which an inputted indication that is provided to the recipient via the stimulator may be based, and an information scheduler configured to control the time at which the information signal is delivered to the stimulator based on the sound analysis signal.

According to another aspect of the present invention, there is a method of delivering an inputted indication to a recipient of a hearing prosthesis via a stimulator of the hearing prosthesis and enhancing hearing via the stimulator, the method comprising enhancing hearing by stimulating tissue of the recipient with the stimulator, determining a delivery time to deliver the inputted indication to the recipient in accordance with analysis of external sound and/or a recipient state, and delivering the inputted indication to the recipient via the stimulator at the determined delivery time.

According to another aspect of the present invention, there is a hearing prosthesis for use by a recipient to enhance hearing, the hearing prosthesis comprises a receiver configured to receive sounds, a sound processor configured to receive an input from the receiver and output a stimulator control signal, a stimulator configured to receive the stimulator control signal and stimulate tissue of the recipient to enhance recipient hearing. According to this aspect, the hearing prosthesis is configured to deliver an inputted indication to the recipient via the stimulator that is not based on the received sounds, and the hearing prosthesis is configured to control a scheduling of the delivery of the inputted indication to the recipient based on at least one of the received sounds and a recipient state.

According to another aspect of the present invention, there is a hearing prosthesis for use by a recipient to enhance hearing, the hearing prosthesis comprises a receiver for receiving an input signal representative of a state external to the hearing system, a sound analyzer for analyzing the input signal received by the receiver and for outputting an input signal analysis signal, an information signal generator for generating an information signal upon which an inputted indication that is provided to the recipient may be based, and an information scheduler for controlling the schedule at which the inputted indication is delivered to the recipient in accordance with the input signal analysis signal.

### BRIEF DESCRIPTION OF THE DRAWINGS

Illustrative embodiments of the present invention will be discussed with reference to the accompanying drawings, in which:

FIG. 1 depicts an exemplary system block diagram of a hearing prosthesis sub-assembly according to an exemplary embodiment of the present invention;

FIG. 2 depicts an exemplary scenario of an input audio signal categorized into different environmental environments;

FIG. 3 depicts an exemplary schedule of inputted indications for scheduling into the scenario depicted in FIG. 2;

FIG. 4 depicts an example of classification of an environment classified into different categories;

FIG. 5 depicts an example of information signal scheduling without the use of priorities with respect to the environment represented in FIG. 4;

FIG. 6 depicts an example of various information signals scheduled into the audio signal;

FIG. 7 depicts a system block diagram of a variation of a hearing prosthesis;

FIG. 8 depicts a system block diagram of a further variation of a hearing prosthesis;

FIG. 9 depicts a block diagram of a further variation of a hearing prosthesis;

FIG. 10 depicts a cochlear hearing prosthesis;

FIG. 11 depicts a perspective view of a cochlear hearing prosthesis usable with some embodiments of the present invention; and

FIG. 12 depicts an exemplary system block diagram of the information scheduler 40 depicting inputs and outputs of the information scheduler 40 according to an embodiment of the present invention.

### DETAILED DESCRIPTION

An embodiment of the present invention includes a hearing prosthesis for use by a recipient to enhance hearing. The hearing prosthesis includes a receiver configured to receive sounds, a sound processor configured to receive an input from the receiver and output a stimulator control signal, a stimulator configured to receive the stimulator control signal and stimulate tissue of the recipient to enhance recipient hearing. The hearing prosthesis is configured to deliver an inputted indication to the recipient via the stimulator that is not based on the received sounds, the hearing prosthesis is configured to control a scheduling (also referred to herein as “timing”) of the delivery of the inputted indication to the recipient based on at least one of the received sounds and a recipient state, as will now be described.

Embodiments of the present invention may be utilized with a hearing prosthesis, which may be a cochlear prosthesis (commonly referred to as cochlear prosthetic devices, cochlear implants, cochlear devices, and the like; simply “cochlear implants” herein). Cochlear implants deliver electrical stimulation to the cochlea of a recipient. As used herein, cochlear implants may be used in combination with other types of stimulation, such as acoustic or mechanical stimulation (sometimes referred to as mixed-mode devices). Embodiments of the present invention may be implemented in any cochlear implant or other hearing prosthesis now known or later developed, including auditory brain stimulators, or implantable hearing prostheses that mechanically stimulate components of the recipient's middle or inner ear, including those that provide vibration to bone tissue of the recipient (e.g., bone conduction devices). Embodiments of the present invention may be implemented in acoustical hearing aids. Embodiments of the present invention may be implemented in any form of hearing aid now known or later developed.

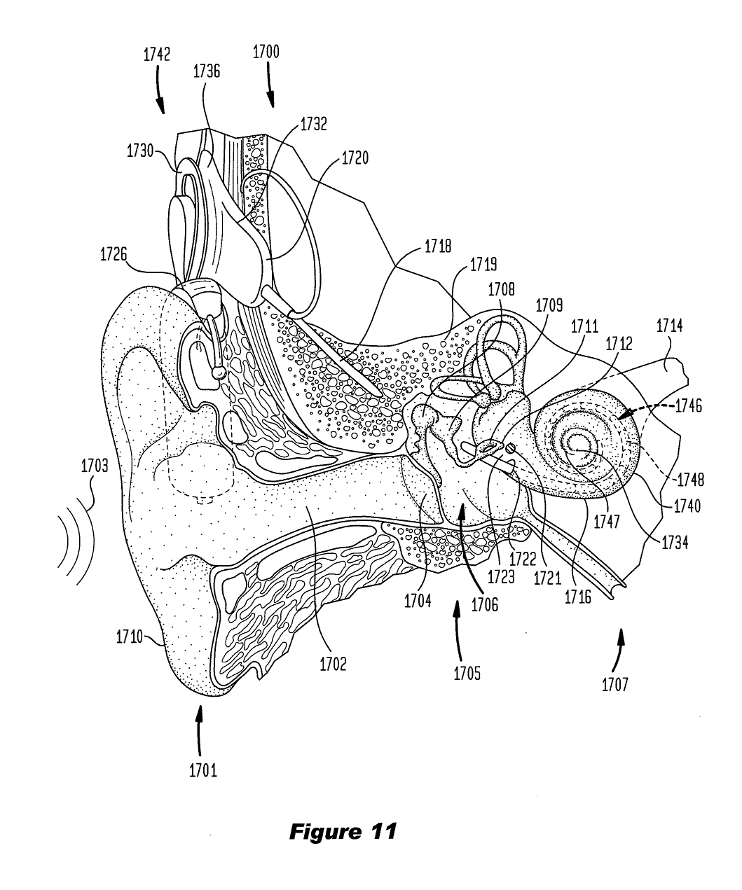

FIG. 11 is perspective view of a cochlear implant, referred to as cochlear implant 1700, implanted in a recipient. The recipient has an outer ear 1701, a middle ear 1705 and an inner ear 1707. Components of outer ear 1701, middle ear 105 and inner ear 1707 are described below, followed by a description of cochlear implant 1700.

In a fully functional ear, outer ear 1701 comprises an auricle 1710 and an ear canal 1702. An acoustic pressure or sound wave 1703 is collected by auricle 1710 and channeled into and through ear canal 1702. Disposed across the distal end of ear cannel 1702 is a tympanic membrane 1704 which vibrates in response to sound wave 1703. This vibration is coupled to oval window or fenestra ovalis 1712 through three bones of middle ear 1705, collectively referred to as the ossicles 1706 and comprising the malleus 1708, the incus 1709 and the stapes 1711. Bones 1708, 1709 and 1711 of middle ear 1705 serve to filter and amplify sound wave 1703, causing oval window 1712 to articulate, or vibrate in response to vibration of tympanic membrane 1704. This vibration sets up waves of fluid motion of the perilymph within cochlea 1740. Such fluid motion, in turn, activates tiny hair cells (not shown) inside of cochlea 1740. Activation of the hair cells causes appropriate nerve impulses to be generated and transferred through the spiral ganglion cells (not shown) and auditory nerve 1714 to the brain (also not shown) where they are perceived as sound.

As shown, cochlear implant 1700 comprises one or more components which are temporarily or permanently implanted in the recipient. Cochlear implant 1700 is shown in FIG. 11 with an external device 1742 which, as described below, is configured to provide power to the cochlear implant.

In the illustrative arrangement of FIG. 11, external device 1742 may comprise a power source (not shown) disposed in a Behind-The-Ear (BTE) unit 1726. External device 1742 also includes components of a transcutaneous energy transfer link, referred to as an external energy transfer assembly. The transcutaneous energy transfer link is used to transfer power and/or data to cochlear implant 1700. As would be appreciated, various types of energy transfer, such as infrared (IR), electromagnetic, capacitive and inductive transfer, may be used to transfer the power and/or data from external device 1742 to cochlear implant 1700. In the illustrative embodiments of FIG. 11, the external energy transfer assembly comprises an external coil 1730 that forms part of an inductive radio frequency (RF) communication link. External coil 1730 is typically a wire antenna coil comprised of multiple turns of electrically insulated single-strand or multi-strand platinum or gold wire. External device 1742 also includes a magnet (not shown) positioned within the turns of wire of external coil 1730. It should be appreciated that the external device shown in FIG. 11 is merely illustrative, and other external devices may be used with embodiments of the present invention.

Cochlear implant 1700 comprises an internal energy transfer assembly 1732 which may be positioned in a recess of the temporal bone adjacent auricle 1710 of the recipient. As detailed below, internal energy transfer assembly 1732 is a component of the transcutaneous energy transfer link and receives power and/or data from external device 1742. In the illustrative embodiment, the energy transfer link comprises an inductive RF link, and internal energy transfer assembly 1732 comprises a primary internal coil 1736. Internal coil 1736 is typically a wire antenna coil comprised of multiple turns of electrically insulated single-strand or multi-strand platinum or gold wire. Positioned substantially within the wire coils is an implantable microphone system (not shown). As described in detail below, the implantable microphone assembly includes a microphone (not shown), and a magnet (also not shown) fixed relative to the internal coil.

Cochlear implant 1700 further comprises a main implantable component 1720 and an elongate electrode assembly 1718 extending from the main implantable component 1720. In embodiments of the present invention, internal energy transfer assembly 1732 and main implantable component 1720 are hermetically sealed within a biocompatible housing. In embodiments of the present invention, main implantable component 1720 includes a sound processing unit (not shown), also referred to as a sound processor, to convert the sound signals received by the implantable microphone in internal energy transfer assembly 1732 to data signals, although in other embodiments, the sound processing unit may be located in the external components of the cochlear implant. Main implantable component 1720 further includes a stimulator unit (also not shown) which generates electrical stimulation signals based on the data signals. The electrical stimulation signals are delivered to the recipient via elongate electrode assembly 1718.

Elongate electrode assembly 1718 has a proximal end connected to main implantable component 1720, and a distal end implanted in cochlea 1740. In an exemplary embodiment, the electrode assembly 1718 is connected to the main implantable component 1720 via a feedthrough component, which may be manufactured according to the embodiments described herein. The feedthrough component permits the maintenance of the hermetic seal of the biocompatible housing just discussed, while permitting electrical signals to pass through the hermetic seal from/to the main implantable component 1720 to/from the electrode assembly 1718. In an exemplary embodiment, the flange brazed to the feedthrough component may be laser welded to the housing to attach the feedthrough to the housing. In embodiments of the present invention, internal energy transfer assembly 1732 and main implantable component 1720 are hermetically sealed within a biocompatible housing.

Electrode assembly 1718 extends from main implantable component 1720 to cochlea 1740 through mastoid bone 1719. In some embodiments electrode assembly 1718 may be implanted at least in basal region 1716, and sometimes further. For example, electrode assembly 1718 may extend towards apical end of cochlea 1740, referred to as cochlea apex 1734. In certain circumstances, electrode assembly 1718 may be inserted into cochlea 1740 via a cochleostomy 1722. In other circumstances, a cochleostomy may be formed through round window 1721, oval window 1712, the promontory 1723 or through an apical turn 1747 of cochlea 1740.

Electrode assembly 1718 comprises a longitudinally aligned and distally extending array 1746 of electrodes 1748, sometimes referred to as electrode array 1746 herein, disposed along a length thereof. Although electrode array 1746 may be disposed on electrode assembly 1718, in most practical applications, electrode array 1746 is integrated into electrode assembly 1718. As such, electrode array 1746 is referred to herein as being disposed in electrode assembly 1718. As noted, a stimulator unit generates stimulation signals which are applied by electrodes 1748 to cochlea 1740, thereby stimulating auditory nerve 1714.

Cochlear implant 1700 may comprises a totally implantable prosthesis that is capable of operating, at least for a period of time, without the need for external device 1742. Therefore, cochlear implant 1700 further comprises a rechargeable power source (not shown) that stores power received from external device 1742. The power source may comprise, for example, a rechargeable battery. During operation of cochlear implant 1700, the power stored by the power source is distributed to the various other implanted components as needed. The power source may be located in main implantable component 1720, or disposed in a separate implanted location.

As noted above, the cochlear implant 1700 includes a sound processor. Other hearing prostheses may also include a sound processor. In an exemplary embodiment of a sound processor, usable in many hearing prostheses, the incoming external sound captured by, for example, a microphone, is digitized by an analogue to digital convertor (AD) and then processed in the digital domain by a digital signal processor (DSP). Digital to analogue (DA) convertors may be used to output an analogue output that is used by or otherwise used to generate output by, for example, a speaker (in the case of an acoustic hearing aid) and/or to that is used to stimulate electrodes via a cochlear implant electrode (in the case of a cochlear implant, such as cochlear implant 1700 described above).

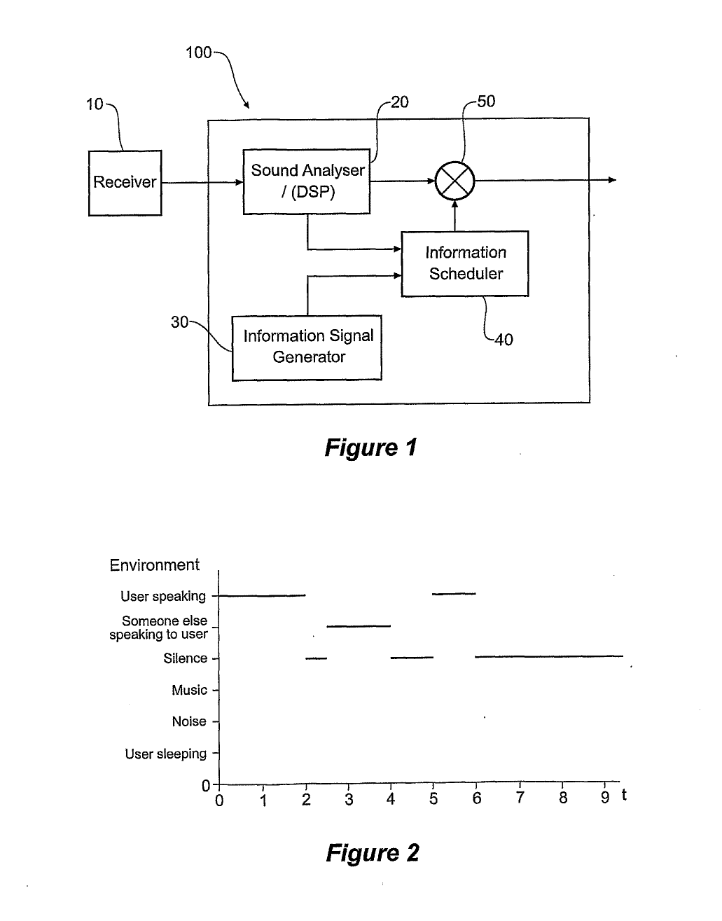

FIG. 1 depicts a hearing prosthesis sub-assembly 100, which may correspond by way of example to part or all of external device 1742, while in other embodiments, some or all of the components of hearing prosthesis sub-assembly 100 may correspond to the implantable component 1720. This latter scenario may be the case, by way of example, in the case of a full-implantable hearing prosthesis where all components, including the receiver 10, are implanted in the recipient. The hearing prosthesis sub-assembly 100 may include a sound processor unit as detailed above. Apart from the sound processing functions, the sub-assembly 100 may also be configured to execute a number of supporting functions. This may include monitoring the hearing prosthesis in which it is a part and informing the recipient of any problems and/or other developments about which the recipient should be aware. Examples of such warnings/developments according to an exemplary embodiment of the present invention may include:
1. Warning the recipient that the battery is going flat;
2. Informing the recipient that a program change has occurred;
3. Informing the recipient that a cable is broken;
4. Informing the recipient that an accessories cable is being connected;
5. Informing the recipient that the telecoil is on; and
6. Informing the recipient that the microphone protection cover needs replacement; etc.

This information may be provided to the recipient in a number of ways, such as, for example, as information signals, including inserting a number of beeps into a sound processing path of the hearing prosthesis.

Examples of these information signals usable in an exemplary embodiment may be:
1. Providing one low tone beep if program 1 becomes active;
2. Providing two low tone beeps if program 2 becomes active;
3. Providing a high tone beep if the battery needs replacement within the hour; and
4. Providing two high tone beeps if the battery is going flat in 10 minutes; etc.

In another exemplary embodiment, a pre-recorded vocal sentence (samples) is provided to the recipient to inform the recipient of the problem/development. In this way, the recipient does not have to remember or otherwise refer to an instruction manual or the like what the different types of beeps indicate.

Along these lines, in an exemplary embodiment, the hearing prosthesis pay provide a vocal message such as:
1. “Please change your microphone protection cover” when the sound processor determines that the sound quality of the incoming microphone signal is poor;
2. “Program 1 active” when program 1 becomes active; and
3. “Your batteries will be flat in one hour” when the battery voltage drops under a certain level; etc.

Alternatively, these vocal messages may be generated in real time (e.g., synthesized) rather than being pre-recorded.

In other embodiments, other ways of indicating to the user a problem/development may be utilized, such as, a chime, a tune, ticking, an alteration to overall sound perceived by the recipient (e.g., the sound may have a sudden increase and/or decrease in volume, thereby permitting the user to continue to perceive (and potentially continue to understand) the sound while also indicating to the user a problem/development.

Hereinafter, such ways of indicating to the user such information by way of hearing and/or perceptual hearing are referred to as “inputted indications.”

In an exemplary embodiment, there is a hearing prosthesis configured such that while the recipient is listening (which, as used herein, includes the scenario of a totally or partially deaf person perceiving sound and/or perceiving sound in some frequencies and/or in one ear and hearing sounds in some frequencies and or in another ear via use of a hearing prosthesis) to sound received by the hearing prosthesis, the inputted indications (e.g., beeps or the vocal messages, etc.) are mixed or otherwise injected into the same signal path of the hearing prosthesis as the signals used to convey sound to the recipient. Other embodiments provide the inputted indications in other ways. Some exemplary embodiments of the present invention includes any device, system, method or algorithm that may be used to provide inputted indications to a user via a hearing prosthesis.

An embodiment of the present invention provides a more user-compatible system of providing the inputted indications to the recipient. In this regard, a recipient might sometimes miss or otherwise fail to comprehend key sounds upon receiving an inputted indication. In some instances, the delivery of the inputted indications may be inputted at an inconvenient time for the recipient. For example, the recipient may be interrupted by receipt of the inputted indications at the moment he or she is trying to understand someone talking directly to him or her. Alternatively, the recipient might be sleeping and not be interested in information that is not time critical (e.g., the recipient may not want to receive the inputted indication of “please change your microphone protection cover” while sleeping). Alternatively, if the recipient is talking, he or she might miss sound information that is provided to him or her at the same time.

In an exemplary embodiment of the present invention, there is are methods, apparatuses and systems that reduces the interruption of normal use of the hearing prosthesis due to the delivery of the inputted indications. In this aspect, the scheduling of the delivery of the inputted indications may be controlled so as to reduce interruptions and/or control the interruptions to times more convenient for the recipient. In an exemplary aspect, this involves analysis of surrounding or external sounds around the recipient (which may include simply analyzing post-processed signals that will be used by the hearing prosthesis), to determine an appropriate delivery time/determine an inappropriate delivery time. In another exemplary embodiment of the present invention, there is a hearing prosthesis that includes an information scheduler to control the delivery of the inputted indications to the recipient in accordance with the results of an analysis of the surrounding sound.

Referring again to FIG. 1, there may be seen a receiver 10 for receiving sounds external to the recipient. Receiver 10 may be any suitable audio receiver, such as a microphone. The sounds received by receiver 10 are provided to sound analyzer 20, which performs one or more suitable signal processing functions to the received sound to enable determination of an appropriate time for delivery of information signals to the recipient. These processes will be described in more detail below.

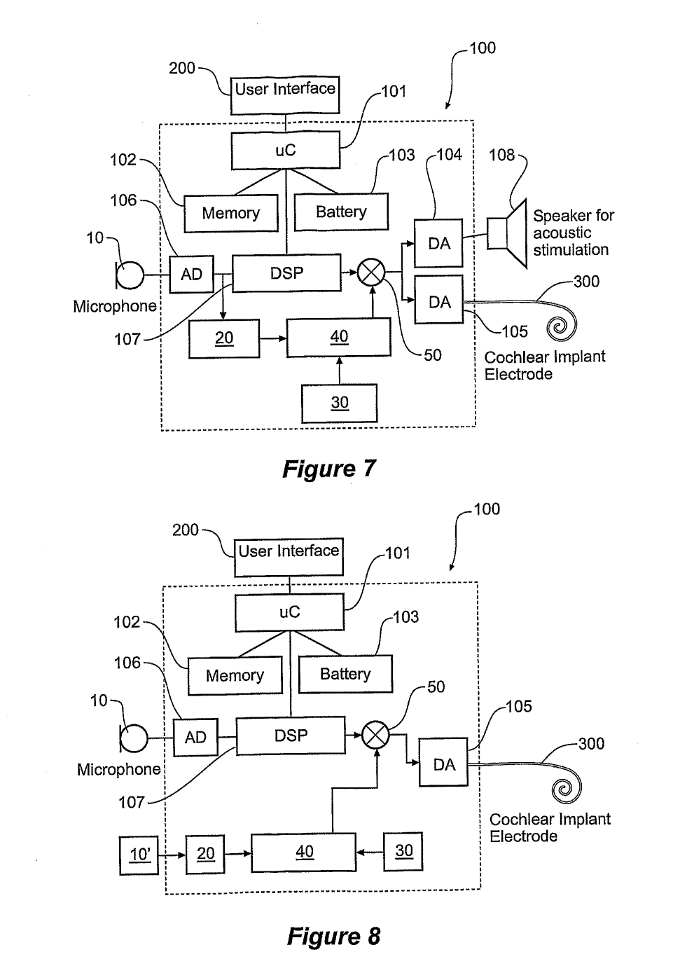

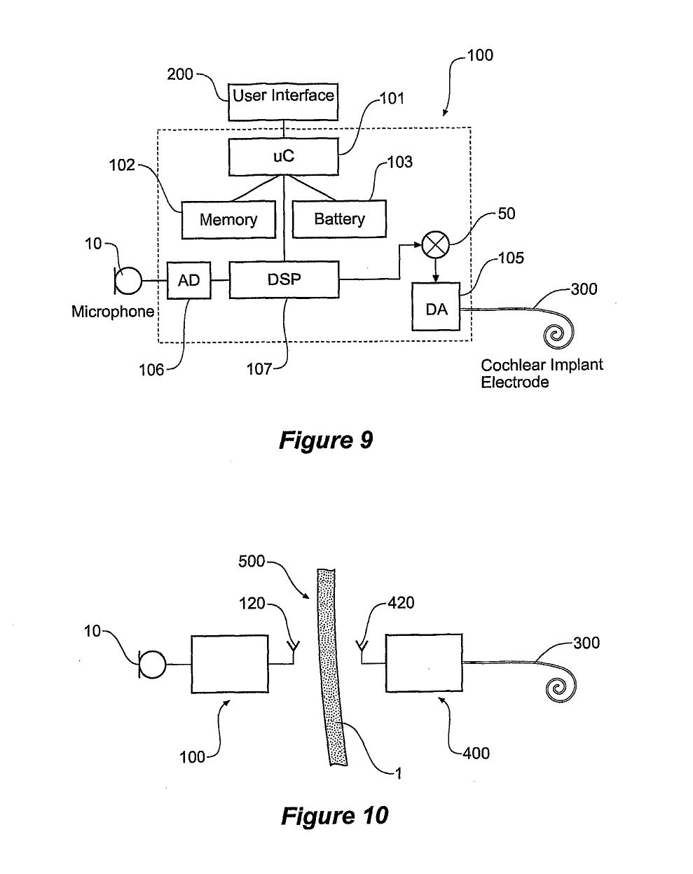

In the embodiment of FIG. 1, the sound analyzer 20 permits the signals from receiver 10 to pass through the sound analyzer 20 so that the signals may later be received by a sound processing unit (not shown), where the sound processing unit may correspond to the sound processing unit described above with respect to FIG. 11. Sound analyzer 20 may also be provided as software in a microprocessor, or a dedicated processing chip. Sound analyzer 20 may be incorporated within the normal signal processing function of a traditional sound processor (as is the case with the embodiment of FIG. 9, described in greater detail below), or may be provided as a separate device (as will be explained in greater detail below with respect to FIGS. 7 and 8).

In an exemplary embodiment, the configuration of FIG. 1 may be integrated into the hearing prosthesis 1700 of FIG. 11, either into the external device 1742, into the implantable component 1720, or the components detailed in FIG. 1 may be divided between the two components or other components of the hearing prosthesis 1700.

Information signal generator 30 generates appropriate information signals for the recipient, which, when used as a basis to provide stimulation to the recipient, results in the inputted indications described above. It is noted that in some embodiments, the information signal generator 30 is a memory device that stores signals indicative of the inputted indications to be delivered to the recipient, while in other embodiments, the information signal generator 30 is a synthesizer device that synthesizes the inputted indications. These information signals may pertain to the status of the hearing prosthesis, such as, for example, a low battery, or may pertain to any other information as will be described further below. Again, as previously described, the inputted indications may be in the form of a series of sounds such as beeps, or actual spoken words, either prerecorded or generated in real time.

In the embodiment depicted in FIG. 1, information signal generator 30 outputs the generated information signals to the information scheduler 40 to control/coordinate the delivery of the information signal/the inputted indications in accordance with the sound analysis signal output from the sound analyzer 20. In an alternate embodiment, signal generator 30 outputs the generated information signals directly to the mixer 50 (described in greater detail below), as opposed to the information scheduler 40. In such an embodiment, the scheduling of the output of the signal generator 30 may be controlled by the information scheduler 40. In yet another embodiment, the output of the signal generator 30 received by the mixer 50 may be held in a queue and/or held in a memory unit that is part of or connected to mixer 50 until a determined time when the inputted indications should be delivered to the recipient.

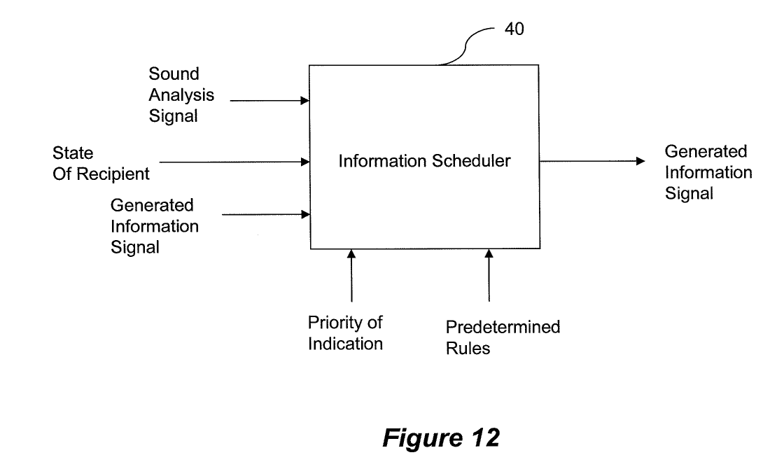

FIG. 12 depicts inputs and outputs of information scheduler 40 in an exemplary embodiment of the present invention. As may be seen, information scheduler 40 receives a sound analysis signal that is outputted from sound analyzer 20. Information scheduler 40 also receives the generated information signal generated by information signal generator 30, although in other embodiments, information scheduler 40 may instead or in addition to this receive a signal indicative of the generated information signal. Information scheduler 40 also receives a state of recipient signal, which provides a state of the recipient (such as whether the recipient is sleeping), as will be described in greater detail below. Further, the information scheduler receives information pertaining to the priority of the various inputted indications that may result from the signal generated by the information signal generator. This information may be stored in a look-up table or the like in the information scheduler and/or accessed in a look-up table or the like stored in a remote memory. The information scheduler analyzes the various inputs (or lack thereof) according to a predetermined set of rules also inputted into the information scheduler (or accessed in a remote component) to determine when to output (or otherwise permit delivery of) the generated information signal so as to deliver an inputted indication to the recipient.

In an exemplary embodiment, rules may be inputted into the information scheduler to address the scenario where several information signals are stored in a queue in the information scheduler 40 for delivery at an appropriate time. The stored information signals may be delivered on a first-in-first-out basis, or alternatively, each information signal may have assigned to it, a priority, which will affect the delivery time of that information signal, as will be described in more detail below.

When the information scheduler 40 determines that it is an appropriate time to deliver an inputted indication, the information scheduler 40 applies the information signal from information signal generator 30 to mixer 50 to deliver the inputted indication to the recipient. Mixer 50 will mix the received information signal with the signal(s) from receiver 10 that are passed through sound analyzer 20. In an alternate embodiment, such signals bypass sound analyzer 20 altogether, and are delivered directly to the mixer 50. The signals from receiver 10 and from information scheduler 40 (or information signal generator 30) are mixed together at mixer 50. The output from the mixer 50 may be directed to a sound processing unit, such as that detailed above with respect to FIG. 11, so that the mixed signals may be converted to data signals for ultimate deliver to a device, such as a stimulator of a cochlear implant, to enhance hearing. In an exemplary embodiment of the invention, the information signal will be mixed into the signal stream from the receiver 10 at an appropriate moment such as when there is silence around the recipient, as determined based on the analysis by the sound analyzer 20. In this way, the inputted indications are less likely to interfere with the normal operation of the hearing prosthesis by the recipient (e.g. interrupting speech) and is more likely to be clearer to the recipient because it is not interfered with by other sound.

In an alternate embodiment of the present invention, instead of mixing the information signal prior to the sound processor unit, the information signal is mixed with the output of the sound processor unit. That is, in an exemplary embodiment, mixer 50 is located downstream from the signal processor, as is depicted by way of example with respect to the embodiments of FIGS. 7 and 8, described in greater detail below. In this regard, whereas with respect to FIG. 1, the information signal from the information signal generator may be the same as or analogous to an audio signal, as it is mixed with other audio signals from receiver 10 at mixer 50, the information signal from the information signal generator 30 in such an alternate embodiment may be the same as or analogous to the data codes outputted by a speech processor, where these data codes are ultimately received by, for example, a stimulator of a cochlear implant. Accordingly, the output of the information signal generator 30 may vary in type depending on where the output is mixed.

In an alternate embodiment, a mixer may not necessarily be used. Instead, the normal signal path may be partially or entirely interrupted so that the information signal may be inserted in the path, after which the normal signal path may be reestablished. By way of example, element 50 may be a switch instead of a mixer. In some embodiments, combinations of these embodiments may be practiced.

More specific features of some embodiments of the present invention will now be described.

Referring back to the embodiment depicted in FIG. 1, the sound analyzer 20 may use an output signal from, for example, a microphone (corresponding to receiver 10) to classify the sound environment in which recipient finds himself or herself. In an exemplary embodiment, such environments may be classified into, by way of example and not by limitation:
1. Recipient speaking;
2. Someone is speaking to the recipient;
3. Recipient is in a silent environment;
4. Recipient is sleeping;
5. Recipient is in a noise environment; and
6. Recipient is listening to music; etc.

There are many ways that sound may be classified in some embodiments of the present invention. In some embodiments, the results of this classification may be used to automatically optimize certain sound processing parameters or switch programs, etc. An example of this is to switch on a directional microphone when in a noisy environment. The algorithms which may be used may extract certain features from a received signal(s) and use a rule-based decision approach. Examples of such systems and/or algorithms are described in various patents and patent applications, and may be used in some embodiments of the present invention to this end. By way of example, such systems and algorithms may correspond to some or all of those disclosed in European Patent Application No. EP0707433 entitled “Hearing Aid” By way of example, an embodiment of the present invention may utilize the method and/or means for detecting a voiceless period based on an analysis of sound received by a sound input means disclosed therein. In embodiments utilizing the method disclosed therein, inputted indications may be delivered to the recipient during periods that are determined to be voiceless.

Another exemplary embodiment of the present invention utilizes some or all of the teachings of U.S. Pat. No. 5,604,812 entitled “Programmable Hearing Aid With Automatic Adaption To Auditory Conditions,” which describes. For example, an exemplary embodiment of the present invention may utilize the an apparatus and/or a method as disclosed in that US patent for analyzing ambient noise conditions and causing the apparatus to perform certain functions (such as activating a directional microphone when in a noisy environment). This method may be used in some embodiments of the present invention to classify ambient or environmental noise conditions.

Another exemplary embodiment of the present invention utilizes some or all of the teachings of U.S. Pat. No. 5,819,217 entitled “Method and System for Differentiating Between Speech and Noise.” For example, an exemplary embodiment of the present invention may utilize the method and/or system as disclosed in that US patent for differentiating between speech and noise by separating an incoming audio signal into frames, evaluating energy levels of selected frames, and/or determining whether the period associated with those frames is noise or speech depending upon the energy evaluation. This method may be used in an exemplary embodiment present invention to, for example, classify different noise conditions to therefore determine an appropriate time for delivering inputted indications to the recipient.

Another exemplary embodiment of the present invention utilizes some or all of the teachings of U.S. Pat. No. 3,909,532 entitled “Apparatus and Method For Determining the Beginning and the End of a Speech Utterance.” For example, an exemplary embodiment of the present invention may utilize the method as disclosed in that US patent for determining the energy of a code word and determining whether it is the beginning or end of an utterance by comparing the code word energy with a threshold. This information can be used in an exemplary embodiment of the present invention to determine periods between speech utterances to, for example, deliver a short inputted indication of high priority to the recipient.

Another exemplary embodiment of the present invention utilizes some or all of the teachings of U.S. Pat. No. 6,009,396 For example, an exemplary embodiment of the present invention may utilize a method of determining when someone is speaking to the recipient as described in that US patent. By using multiple microphones, the direction of the incoming sound can be determined. It can be assumed that when speech is coming from in front of the recipient this is a person talking to the recipient. When speech is coming from the side or behind the recipient, this can be considered to be less likely to be someone talking to the recipient.

The state of the recipient sleeping can be detected in several ways using sensors. One method involves measuring the body temperature of the recipient. According to an exemplary embodiment of the present invention, it is assumed that a slight temperature drop in the brain occurs when sleeping. This temperature drop may be measured in the auditory canal. U.S. Pat. No. 4,297,685 discloses such a method that may be used in such an embodiment. In an exemplary embodiment, a temperature sensor could be added to the implantable component and/or the external device of the hearing prosthesis.

Another way of determining that the recipient is asleep, or at least resting, that may be used in an alternate embodiment of the present invention is to measure the orientation of the body. When the body is in a horizontal position for a while it can be assumed that the recipient is resting or sleeping. The orientation of the body can be measured using a gravity sensor. In an exemplary embodiment, a MEMS based accelerometers which are small and can be integrated into the implantable component and/or the external device. An example of such a small accelerometer that may be used in an exemplary embodiment is described in U.S. Pat. No. 4,711,128.

In an exemplary embodiment, other forms of environment may be determined using hidden Markov models, such as those described in U.S. Pat. No. 6,862,359.

Other features may also be used to control the delivery of the inputted indications. For example, a hearing prosthesis according to an exemplary embodiment of the present invention could be made to adapt to what the recipient finds acceptable or unacceptable. According to an exemplary embodiment, an input feature is included in a hearing prosthesis according to an embodiment of the present invention through which the recipient can indicate that he is annoyed by the inputted indications given by the prosthesis or otherwise does views the scheduling of delivery of the inputted indications to be inconvenient. For example, an “annoyed” button can be included in the hearing prosthesis. When the recipient is interrupted while listening to someone else talking and is annoyed by this, the recipient can actuate the “annoyed” button. The hearing prosthesis will now adapt its scheduler rules not to interrupt the recipient again in this environment.

In yet another embodiment of the present invention, a clock may be integrated into the hearing prosthesis and the inputted indication delivery may be based on scheduling governed by the clock. For example, a recipient using a totally implantable hearing prosthesis may elect not to get a “battery low” inputted indication be delivered to him or her in the middle of the night. In this application, the recipient could set the hearing prosthesis to not to deliver this particular inputted indication to the recipient between for example, 10 p.m. and 8 a.m.

FIG. 2 depicts an exemplary time graph representative of input audio signal(s) that has been analyzed and classified into different categories using any suitable analysis and/or classification technique including one or more of the techniques described in the above-referenced patents. Shown in FIG. 2 is a temporally-separated input audio signal, the input audio signal being classified into “recipient speaking”, “someone else is speaking to the recipient” and “silence” categories. The input audio signal has been classified by the sound analyzer 20. It is noted that in other embodiments, FIG. 2 may represent a temporally-separated output signal from a speech processor, classified by the sound analyzer 20.

In some embodiments, the sound analyzer 20 classifies in a continuous manner. As shown in FIG. 2, at every moment in time the hearing prosthesis attributes a certain environment or classification to the input audio signal (or output signal).

As previously described, the information scheduler 40 (see FIG. 1) decides on when to mix information signals with mixer 50 he sound processing path or otherwise when to provide the inputted indications to the recipient. In an exemplary embodiment, information scheduler 40 compares an incoming information signal queue with sound environment. In a further aspect of the invention, a rule-based system may be used to determine when to mix the information signal at mixer 50. An example of a prioritization table usable in such a rules-based system is shown below in Table 1.

TABLE 1

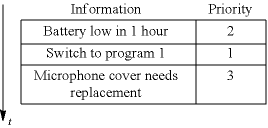

In this exemplary embodiment, the incoming information signal queue contains the information signals that will be mixed at mixer 50, thereby communicating the associated inputted indications to the recipient in a prioritized manner. The queue can be of any length and is only limited by the amount of memory available in the hearing prosthesis. The queue can be more advanced and include additional information such as the number of times a message needs to be repeated or specific scheduling information such as preference to inform something early in the morning or during the charging process.

In one embodiment, to determine when to mix the information signals at mixer 50, the information scheduler 40 uses a rules-based system. In an exemplary embodiment, the information scheduler 40 checks a number of rules one by one over and over again. In other embodiments, other algorithms may be utilized. When the rules result in a decision to provide the inputted indications to the recipient, the respective information signal is mixed in with the other signals at mixer 50.

The following is an exemplary set of rules that may be used in some embodiments of the present invention:
1. If information available of priority 1 and recipient is not speaking, provide the inputted indication(s) to the recipient;
2. If information available of priority 2 and recipient not speaking and someone else is not speaking, provide the inputted indication(s) to the recipient; and
3. If information available of priority 3 and silence for longer than 2 seconds, provide the inputted indication(s) to the recipient.

An exemplary embodiment of a hearing prosthesis utilizing the exemplary rules just detailed, in combination with the incoming information queue in Table 1, when placed in the sound environment as represented in FIG. 2, the following results may be seen.

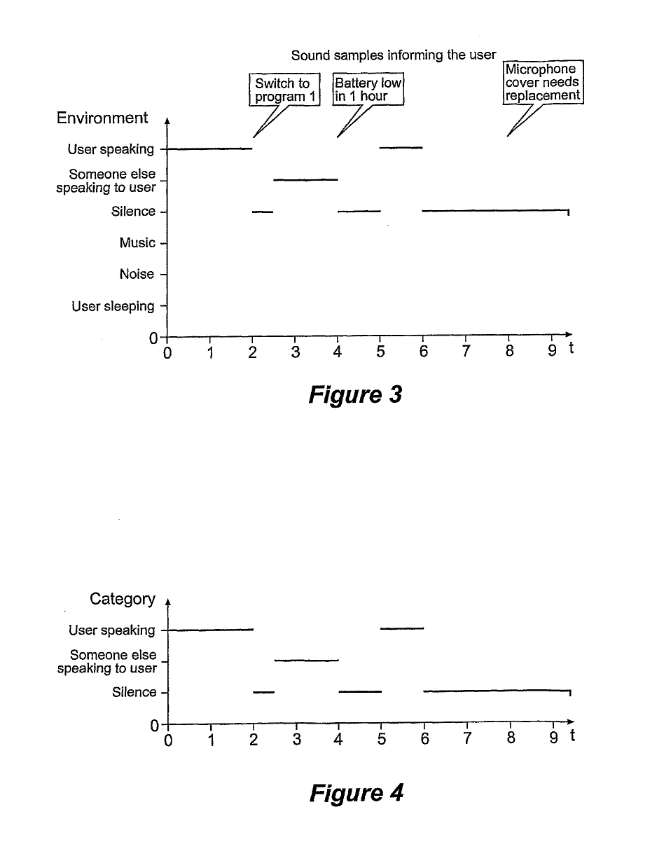

Referring to FIG. 3, at moment 2 in time, the first rule (with reference to the above rules) is valid and the message of priority 1 is processed and removed from the information queue. At moment 4 the second rule is valid and the message of priority 2 is processed and removed from the information queue. At moment 8, the 3r^{d} rule is valid and the message of priority 3 is processed and removed from the information queue.

The following pseudo code provides an example for carrying out the method described above in relation to FIG. 3:

timer = 0
silence = 0
// endless loop
Begin Loop

// silence counter
If (Environment == “Silence”) then

silence = silence + 1

Else

silence = 0

End if
// implementation of the first rule
If (Information_Queue_Priority_1 != null) AND (Environment !=
“Recipient speaking”) then

Process(Information_Queue_Priority_1)

End if
// implementation of the second rule
If (Information_Queue_Priority_2 != null) AND (Environment !=
“Recipient speaking”)
AND (Environment != “Someone else speaking to the recipient”)
then

Process(Information_Queue_Priority_2)

End if
// implementation of the third rule
If (Information_Queue_Priority_3 != null) AND (silence == 2) then

Process(Information_Queue_Priority_3)

End if
// timer counter
timer = timer + 1

End Loop

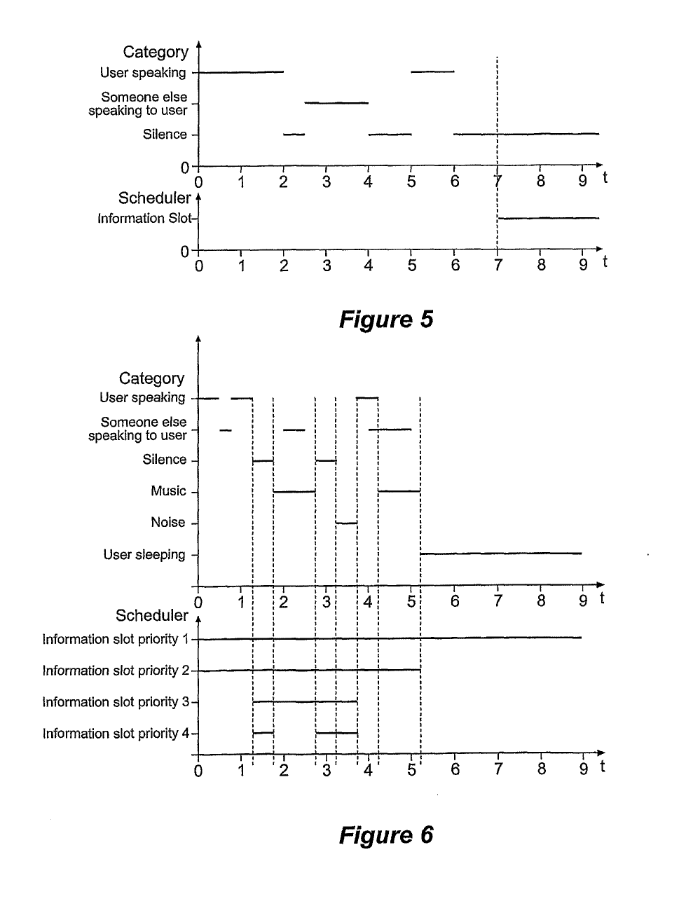

In another example in which the use of priorities is not made is presented with respect to FIGS. 4 and 5. Here, the sound analyzer 20 classifies the sound into different categories in a continuous way as previously described, as may be understood in view of FIG. 4.

At the same time, the information scheduler 40 identifies moments at which inputted indications may be proved to the recipient, such as, for example, at moments when there is more than 1 second of silence. FIG. 5 shows where the information scheduler 40 has identified an information slot for delivery of the inputted indications to the recipient. This is shown at time 7 seconds. At that moment, the category silence was active for more than 1 second.

Yet another example, in which priorities are assigned, will now be described with reference to FIG. 6. In an exemplary embodiment of the present invention in which information is prioritised, information scheduler 40 determines slots for each type of inputted indication. With respect to the scenario depicted in FIG. 6, in such an exemplary embodiment, the information scheduler 40 has determined that inputted indications corresponding to priority 1 can be given at all times. Conversely, inputted indications of priority 2 (lower priority than the information of priority 1) can be given at times during which the recipient is not sleeping. In contrast to both priority 1 and priority 2, Priority 3 is for when the recipient is not talking or being talked to (silence, only noise or music is being received by receiver 10, etc.). Priority 4 does not interrupt music, but will permit an interruption during noise and silence.

FIG. 7 shows a block diagram of a hearing prosthesis sub-assembly 100 for a hybrid hearing prosthesis combining a cochlear implant with an acoustical hearing aid. Shown in FIG. 7 is receiver 10 (in this case, provided by a microphone), analogue-to digital (AD) converter 106 for converting the analogue sound signal outputted by receiver 10 to a digital signal for processing by digital signal processor (DSP) 107, which may correspond to the sound processor unit detailed above with respect to FIG. 11. DSP 107 generates electrical signals that are representative of sound received by receiver 10. These generated electrical signals are ultimately provided to a stimulator 105 (described in greater detail below) which converts the generated electrical signals to analogue stimulation signals for delivery by electrode array 300. In the embodiment depicted in FIG. 7, the generated signals are also ultimately utilized by speaker 108 to provide acoustic stimulation to the recipient.

Also shown is recipient interface block 200 which allows the recipient to control the hearing prosthesis sub-assembly 100, microcontroller 101 which controls the various communications within sound processor 100, memory 102 and power supply or battery 104. The recipient “annoyed” button previously described may be provided in the recipient interface 200.

The device depicted in FIG. 7 is depicted in terms of a totally implantable hearing prosthesis, where the receiver 10 and the user interface 200 are implanted in the recipient (the user interface 200 is positioned and configured such that the recipient may actuate a button on the user interface by pressing on his or her skin). Speaker 108 may be located in the outer ear, and may communicate with the hearing prosthesis sub-assembly 100 via an RF communications link or a percutaneous lead, as depicted in FIG. 7, etc. In other embodiments of the present invention, the receiver 10 and/or the user interface 200 and/or some of the components of hearing prosthesis sub-assembly 100 (e.g., some or all of components 101, 102, 103, 106, 107, 20, 30, 40, 50) may be located in an external device, and the remaining components may be located in an implanted component.

In the embodiment depicted in FIG. 1, sound analyzer 20 receives the digitized audio signal for analysis as previously described. In some embodiments, the sound analysis as previously described may be integrated with and provided by DSP 107, without a separate sound analysis block 20, as will be discussed in greater detail below. Information signal generator 30 generates the information signals pertaining for example, to system status, and information scheduler 40 controls the integration of the information signals received from information signal generator 30 in accordance with signals received from sound analyzer 20, via mixer 50. According to the embodiment depicted in FIG. 8, the information signals are outputted at times deemed appropriate as described previously. In the particular embodiment represented by FIG. 7, two possible signal outputs are available. The first is via speaker 108 to provide acoustic stimulation, and the second is via cochlear implant electrode assembly 300 for direct electrical stimulation to the recipient's auditory nerves within the recipient's cochlea. Both of these outputs are in analogue form, having been converted from digital signals to analogue signals by DA converters 104 and 105 (DA 105 being a part of a stimulator, and when applied to a hearing prosthesis with an external device and an implanted component, DA 105 is part of a stimulator/receiver, such as may be utilized in a cochlear implant). It will be understood however, that in some embodiments, one or the other output configurations is provided. For example, in one embodiment, stimulation is provided only via cochlear implant electrode assembly 300, and not by any acoustic device, as is depicted by way of example in FIG. 8.

Still referring to FIG. 8, the sound input for sound analysis may be provided by a dedicated receiver 10′ instead of from the receiver 10 used to receive receiving sound input for electrical stimulation of the recipient. As may be seen in FIG. 8, receiver 10′ provides an input directly to sound analyzer 20. In this case, sound analyzer 20 may have its own dedicated DA conversion, or the input of receiver 10′ will be converted by the DA block 106 before input to sound analyzer 20. FIG. 8 depicts an embodiment where the output from hearing prosthesis sub-assembly 100 is delivered solely through cochlear electrode assembly 300. In yet another embodiment, the information signals generated by information signal generator 30 may be used to provided inputted indications to the recipient through an audio speaker 108, as may be seen in FIG. 7, while the sound received by microphone 10 is provided to the recipient via electrode assembly 300 (or visa-versa), with the inputted indications being timed to be provided at the most appropriate time so as not to interfere with stimulation, even though the two paths are separate.

FIG. 9 depicts an alternate embodiment in which the functionality of the sound analyzer is provided by the DSP block 107, the functionality of the information signal generator 30 of FIGS. 1, 7 and 8 is provided by memory 102 alone and/or in combination with microprocessor/microcontroller 101 and/or by microprocessor/microcontroller 101 alone, and the functionality of the information scheduler 20 is provided by microprocessor/microcontroller 101. In this arrangement, separate blocks for these elements may not necessarily be utilized. As used herein, the phrase sound analyzer encompasses any part, any combination of parts and/or sub-part of a hearing prosthesis or comparable device that performs the function of a sound analyzer as detailed herein. Accordingly, by way of example, a sound analyzer encompasses a sound processor unit that processes sound for use in a cochlear implant where the sound processor unit also performs the function of a sound analyzer. As used herein, the phrase information signal generator encompasses any part, any combination of parts and/or sub-part of a hearing prosthesis or comparable device that performs the function of a signal generator as detailed herein. Accordingly, by way of example, a signal generator encompasses a sound processor unit that processes sound for use in a cochlear implant where the sound processor unit also performs the function of a signal generator. As used herein, the phrase information scheduler encompasses any part, any combination of parts and/or sub-part of a hearing prosthesis or comparable device that performs the function of an information scheduler as detailed herein. Accordingly, by way of example, an information scheduler encompasses a microchip that controls all or part of a cochlear implant where the microchip also performs the function of an information scheduler.

More specifically, while the embodiment of FIG. 1 depicts a separate information signal indicative of the inputted indications being delivered from information scheduler 40 (or, in an alternate embodiment, from information signal generator 30) to mixer 50, in the embodiment of FIG. 9, a sound processor unit (DSP block 107) receives signal(s) from receiver 10. The sound processor 1220 and/or the microcontroller 101 includes logic that determines whether it is an appropriate time to provide the recipient with the inputted indications, which may be provided by accessing data stored in and/or accepting data generated by information signal generator (part of one or more of the components depicted in FIG. 9). Such logic may accomplish the function of the information scheduler 40 as detailed above with respect to FIG. 1. Upon a determination that it is an appropriate time to deliver the inputted indications, the sound processor unit (DSP 107) outputs signal(s) that may be used by the hearing prosthesis to provide the inputted indications to the user. Such may be accomplished by providing to the sound processor signal(s) that the signal processor may process (e.g., mixed into the signal path in accordance with the embodiment depicted in FIG. 1), or by combining the output of the sound processor unit with other signals, or by the switching technique detailed above. Any process, system, method or algorithm that will permit the hearing prosthesis to provide the recipient with the inputted indications may be used in some embodiments of the present invention.

It is noted that while the embodiment of FIG. 9 depicts a mixer 50, in other embodiments, no mixer 50 is present. The embodiment of FIG. 9 may provide for later modification of the sub-assembly 100 and/or for production flexibility, in the event that it is desired to mix signals at mixer 50. In the embodiment of FIG. 9, the output of the hearing prosthesis is by way of cochlear implant electrode assembly 300, although, the output could be to an audio speaker or both electrode and audio speaker as previously described, and/or to other stimulating devices. In this regard, the phrase stimulator includes any artificial device that provides stimulation to tissue of the recipient to enhance hearing.

It will be appreciated that any other combination of the above variations is also possible.

It will also be appreciated that FIGS. 7-9 are representative only.

FIG. 10 depicts an example of a cochlear implant in which embodiments of the present invention may be utilized. Shown is a cochlear implant 500 comprising a hearing prosthesis sub-assembly 100 which corresponds to an external device and an implantable component 400 the implantable component 400 includes a stimulator configured to receive signals from external device 100 transmitted through the recipient's tissue 1 and convert those signals into stimulating energy delivered to the cochlea of the recipient via cochlear implant electrode array 300. As shown, implantable component 400 with associated electrode assembly 300 is implanted into the recipient. The hearing prosthesis sub-assembly 100 and implantable component 400 communicate through the recipient's tissue 1 (in this case, the scalp of the recipient behind the recipient's head) transcutaneously by wireless communications.

Hearing prosthesis sub-assembly 100 receives input sound signals from about the recipient via receiver 10 (e.g. a microphone), which are then processed as described above, including the scheduling of the information signals, and applied to a D/A converter. The input sound signal is processed to provide a stimulation signal that is representative of the input sound signal for delivery to the recipient. The hearing prosthesis sub-assembly 100 may include a mixer as described above for mixing in the information signals generated by the information signal generator with the generated stimulation signal at times as controlled by the information scheduler as previously described. The hearing prosthesis sub-assembly 100 may alternatively be configured such that a DSP contained therein may instead produce the information signals, as detailed above.

The output of the DSP contained in the sub-assembly 100, either mixed with the information signal by a mixer or containing the information signal, is provided to transmitter 120 which then transmit the signals wirelessly to corresponding receiver 420 of implantable component 400, through tissue 1. The received signals are then converted into signals (for example, electrical or photonic) and applied directly to auditory nerves in the recipient's cochlea by electrode assembly 300.

In an alternative embodiment of the present invention, the input received by a receiver need not be sound signals. Such an embodiment may be utilized with, for example, the embodiment depicted in FIG. 8, where receiver 10 receives sound input and receiver 10′ receives an alternative signal. The input could be a signal representative of a state of the recipient, hereinafter collectively referred to a recipient state. For example, this could be a state of wakefulness, or an orientation of the recipient, or a state of speaking by the recipient. For example, if the recipient is determined to be asleep, it may be that the processor does not issue the information signal unless it is urgent. This state may be determined by measuring the recipient's body temperature (in which the receiver will be a sensor and in particular, a thermometer), or by determining that the recipient has assumed a horizontal orientation, in which case the receiver may be a gravity sensor, as previously described. The state analyzer will then generate a general input signal analysis signal which is then provided to the information scheduler for controlling the time of delivery of the information signal in accordance with the input signal analysis signal. With respect to FIG. 8, in such an embodiment, the sound analyzer 20 may not necessarily be utilized, as the receiver 10′ may receive a signal that may be utilized to determine the state external to the recipient (and thus it may not be needed to analyze sound).

As may be seen in FIG. 8, in some embodiments, the hearing prosthesis sub-assembly 100 may have multiple receivers. In some embodiments, one or more of the receivers may be a microphone to receive sound, and one or more others may be, for example, a thermometer and/or a gravity sensor. In such an embodiment, a state analyzer may be used to analyze the state of the recipient.

The various aspects of the present invention may be used in some types of hearing prostheses, including one as described in International Patent Application No. PCT/AU96/00403 (W097/01314) entitled “Apparatus And Method Of Controlling Speech Processors And For Providing Private Data Input Via The Same”. This application describes a hearing aid device that receives ambient sounds as well as voice commands from the recipient to control aspects of the operation of the hearing prosthesis. The prosthesis can also deliver messages to the recipient, including various preset alarms, as well as customised recorded messages by the recipient. Various embodiments of the present invention may be combined with various embodiments described in this mentioned application to provide a recipient-friendly or convenient system of delivering messages, including inputted indications to the recipient.

In an exemplary embodiment of the present invention, there is a hearing prosthesis for use by a recipient, the hearing prosthesis comprises a receiver for receiving sounds external to the recipient, a sound analyzer for analyzing the sounds received by the receiver and for outputting a sound analysis signal, an information signal generator for generating an information signal for delivery to the recipient, and an information scheduler for controlling the time at which the information signal is delivered to the recipient, in accordance with the sound analysis signal.

In an exemplary embodiment as described above and/or below, the information scheduler causes the information signal to be delivered to the recipient when the sound analysis signal indicates that there is silence about the recipient. In another exemplary embodiment as described above and/or below, the information scheduler causes the information signal to be delivered to the recipient after the sound analysis signal indicates that the recipient has completed an utterance. In another exemplary embodiment as described above and/or below, the information signal is assigned a priority and the time at which the information signal is delivered to the recipient is determined in accordance with both the sound analysis signal and the assigned priority.

In another exemplary embodiment as described above and/or below, the information signal is delivered to the recipient in accordance with a set of rules. In another exemplary embodiment as described above and/or below, the information signal relates to a status of the medical implant system. In another exemplary embodiment as described above and/or below, the hearing prosthesis is a cochlear implant system.

In another embodiment of the present invention, there is a method of delivering an information signal to a recipient via a hearing prosthesis. The method includes determining a delivery time to deliver the information signal to the recipient in accordance with an analysis of external sound, and delivering the information signal to the recipient at the determined delivery time. In another exemplary embodiment as described above and/or below, the information signal is delivered to the recipient when the analysis of the external sound indicates that there is silence about the recipient. In another exemplary embodiment as described above and/or below, the information signal is delivered to the recipient after the analysis of the external sound indicates that the recipient has completed an utterance. In another exemplary embodiment as described above and/or below, the method further comprises assigning a priority to the information signal and determining the delivery time in accordance with the analysis of the external sound and the assigned priority. In another exemplary embodiment as described above and/or below, the method further comprises determining the delivery time in accordance with the analysis of the external sound and in accordance with a set of rules.

According to another embodiment of the present invention, there is a hearing prosthesis, comprising a hearing prosthesis sub-assembly according to one or more embodiments as described herein, and a signal output stimulator for providing the processed sound to the recipient. In another exemplary embodiment as described above and/or below, the signal output stimulator is a cochlear implant electrode. In another exemplary embodiment as described above and/or below, the signal output stimulator is a speaker.

According to another embodiment of the present invention, there is provided a cochlear implant system for implanting in a recipient. The cochlear implant system comprises a receiver for receiving a sound signal external to the recipient, a sound analyzer for analyzing the sound signal received by the receiver and for outputting a sound analysis signal, an information signal generator for generating an information signal for delivery to the recipient, an information scheduler for controlling the time at which the information signal is delivered to the recipient, in accordance with the sound analysis signal, a signal processor for processing the received sound signal and for generating a stimulation signal representative of the received sound signal, a mixer for mixing the information signal and the stimulation signal as controlled by the information scheduler, and a transmitter for transmitting the stimulation signal mixed with the information signal. The system further comprises a stimulator comprising a receiver for receiving the stimulation signal mixed with the information signal and for delivering the stimulation signal mixed with the information signal to a cochlear stimulation electrode for stimulation of auditory nerves of the recipient's cochlea.

In another exemplary embodiment of the present invention, there is a hearing system for use by a recipient, hearing system comprises a receiver for receiving an input signal representative of a state external to the hearing system, a sound analyzer for analyzing the input signal received by the receiver and for outputting an input signal analysis signal, an information signal generator for generating an information signal for delivery to the recipient, and an information scheduler for controlling the time at which the information signal is delivered to the recipient, in accordance with the input signal analysis signal. In another exemplary embodiment as described above and/or below, the input signal is a signal indicative of a state of wakefulness of the recipient. In another exemplary embodiment as described above and/or below, the input signal is a signal indicative of an orientation of the recipient. In another exemplary embodiment as described above and/or below, the input signal is a sound signal external to the recipient.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgement of any form of suggestion that such prior art forms part of the common general knowledge.

The invention described and claimed herein is not to be limited in scope by the specific preferred embodiments herein disclosed, since these embodiments are intended as illustrations, and not limitations, of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

1. A hearing prosthesis for use by a recipient to enhance hearing, the hearing prosthesis comprising:
a receiver configured to receive sounds external to the recipient;
a stimulator configured to stimulate tissue of the recipient;
a sound analyzer configured to analyze the sounds received by the receiver, the sound analyzer further being configured to output a sound analysis signal indicative of the analyzed sounds;
an information signal generator configured to output an information signal upon which an inputted indication that is provided to the recipient via the stimulator may be based; and
an information scheduler configured to control the schedule at which the information signal is delivered to the stimulator based on the sound analysis signal.

2. The hearing prosthesis as claimed in claim 1, wherein the information scheduler is configured to cause inputted indication to be provided to the recipient when the sound analysis signal indicates that there is silence about the recipient.

3. The hearing prosthesis as claimed in claim 1, wherein the information scheduler is configured to cause the inputted indication to be provided to the recipient when the sound analysis signal indicates that the recipient has completed an utterance.

4. The hearing prosthesis as claimed in claim 1, wherein a priority is assigned to the information signal and wherein the information scheduler is configured to determine a schedule at which the inputted indication is to be provided to the recipient based on both the sound analysis signal and the assigned priority.

5. The hearing prosthesis as claimed in claim 1, wherein the information scheduler is configured to cause the inputted indication to be provided to the recipient in accordance with a set of predetermined rules stored in the hearing prosthesis.

6. The hearing prosthesis as claimed in claim 1, wherein, the information signal relates to a status of the hearing prosthesis.

7. The hearing prosthesis as claimed in claim 6, wherein, the hearing prosthesis is a cochlear implant.

8. A method of delivering an inputted indication to a recipient of a hearing prosthesis via a stimulator of the hearing prosthesis and enhancing hearing via the stimulator, the method comprising:
enhancing hearing by stimulating tissue of the recipient with the stimulator;
determining a delivery schedule to deliver the inputted indication to the recipient in accordance with analysis of sound received by the hearing prosthesis and/or a recipient state; and
delivering the inputted indication to the recipient via the stimulator at the determined delivery schedule.

9. The method as claimed in claim 8, wherein the inputted indication is delivered to the recipient when analysis of the sound received by the hearing prosthesis indicates that there is silence about the recipient.

10. The method as claimed in claim 8, wherein the inputted indication is delivered to the recipient after the analysis of the sound received by the hearing prosthesis and/or the recipient state indicates that the recipient has completed an utterance.

11. The method as claimed in claim 8, further comprising assigning a priority to the inputted indication with respect to other inputted indications and determining the delivery schedule in accordance with the analysis of (i) the sound received by the hearing prosthesis and/or the recipient state and (ii) the assigned priority.

12. The method as claimed in claim 8, further comprising:
determining the delivery schedule in accordance with the analysis of (i) the sound received by the hearing prosthesis and/or the recipient state and (ii) a set of predetermined rules stored in the hearing prosthesis.

13-16. (canceled)

17. A hearing prosthesis for use by a recipient to enhance hearing, the hearing prosthesis comprising:
a receiver for receiving an input signal representative of a state external to the hearing system;
a sound analyzer for analyzing the input signal received by the receiver and for outputting an input signal analysis signal;
an information signal generator for generating an information signal upon which an inputted indication that is provided to the recipient may be based; and
an information scheduler for controlling the schedule at which the inputted indication is delivered to the recipient in accordance with the input signal analysis signal.

18. The processor as claimed in claim 17, wherein the input signal is a signal indicative of a state of wakefulness of the recipient.

19. The processor as claimed in claim 17, wherein the input signal is a signal indicative of an orientation of the recipient.

20. The processor as claimed in claim 17, wherein the input signal is a sound signal external to the recipient.

21. The hearing prosthesis as claimed in claim 1, wherein the information scheduler is configured to cause the inputted indication to be provided to the recipient when the sound analysis signal indicates that the recipient is not speaking and the sound analysis signal indicates that someone else is not speaking.

22. The hearing prosthesis as claimed in claim 1, wherein the information scheduler is configured to cause the inputted indication to be provided to the recipient when the sound analysis signal indicates that the recipient is not speaking.

23. The hearing prosthesis as claimed in claim 1, wherein:
the information signal generator is configured to output a plurality of different information signals upon which respective plurality of inputted indications that are provided to the recipient via the stimulator may be based; and
the information scheduler is configured to recognize that a first of the information signals corresponds to an information signal of a higher predetermined priority than a second of the information signals.

24. The hearing prosthesis as claimed in claim 23, wherein the information scheduler is configured to:
cause a first inputted indication to be provided to the recipient based on the first information signal when the sound analysis signal indicates that the recipient is not speaking; and
cause a second inputted indication to be provided to the recipient based on the second information signal when the sound analysis signal indicates that the recipient is not speaking and the sound analysis signal indicates that someone else is not speaking.

25. The hearing prosthesis of claim 24, further comprising a sensor configured to determine whether the recipient is lying down, wherein the sensor is further configured to output the signal indicative of the recipient state.

26. The hearing prosthesis of claim 1, further comprising:
a receiver configured to receive a signal indicative of a recipient state,
wherein the information scheduler is configured to cause the inputted indication to be provided to the recipient when the signal indicative of the recipient state indicates that the recipient is sleeping.

27. The hearing prosthesis of claim 1, further comprising:
a user interface configured to receive an input from the recipient indicative of a desire by the recipient to not receive at a future time a first inputted indication provided to the user at a first scheduling determined by the information scheduler,
wherein the hearing prosthesis is configured to prevent the first inputted indication from again being provided to the user at the first timing.

28. The hearing prosthesis as claimed in claim 7, wherein the hearing prosthesis is a hybrid device comprising an acoustical hearing aid and a cochlear implant.

29. The method as claimed in claim 8, wherein the stimulator is a stimulator of a cochlear implant.

30. The method as claimed in claim 8, wherein the stimulator is a speaker system of an acoustic hearing aid.

31. The method as claimed in claim 8, further comprising:
providing a first inputted indication to the recipient when the analysis of the sound received by the hearing prosthesis and/or the recipient state indicates that the recipient is not speaking, and
providing a second inputted indication to the recipient when the analysis of the sound received by the hearing prosthesis and/or the recipient state indicates that the recipient is not speaking and the sound analysis signal indicates that someone else is not speaking.

32. The method as claimed in claim 8, further comprising:
receiving an input from the recipient indicative of a desire by the recipient to not receive at a future time the delivered inputted indication at the determined deliver schedule.

33. A hearing prosthesis for use by a recipient to enhance hearing, the hearing prosthesis comprising:
a receiver configured to receive sounds;
a sound processor configured to receive an input from the receiver and output a stimulator control signal;
a stimulator configured to receive the stimulator control signal and stimulate tissue of the recipient to enhance recipient hearing,
wherein the hearing prosthesis is configured to deliver an inputted indication to the recipient via the stimulator that is not based on the received sounds, and
wherein the hearing prosthesis is configured to control a scheduling of the delivery of the inputted indication to the recipient based on at least one of the received sounds and a recipient state.

34. The hearing prosthesis of claim 33, wherein the hearing prosthesis is a cochlear implant.
